(19) United States
(12) Patent Application Publication
(10) Pub. No.: US 2006/0165640 A1
(43) Pub. Date: Jul. 27, 2006

(54) Cosmetic composition comprising at least one alkoxylated alcohol ester and at least one apolar oil

(76) Inventors: Caroline Lebre, Thiais (FR); Audrey Ricard, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/320,637

(22) Filed: Dec. 30, 2005

Related U.S. Application Data
(60) Provisional application No. 60/642,979, filed on Jan. 12, 2005.

(30) Foreign Application Priority Data
Dec. 30, 2004 (FR) 0453263

(51) Int. Cl. A61K 8/35; A61K 8/36

(52) U.S. Cl. 424/70.22; 424/70.31

(57) ABSTRACT

The present disclosure relates to a composition comprising at least one ester derived from at least one alkoxylated alcohol and at least one carboxylic acid; and at least one apolar oil, wherein when the composition forms a deposit, the deposit has a mean gloss measured at 600 of greater than or equal to 45 out of 100. This composition may be used as a care and/or makeup product for keratin materials, such as, e.g., the skin, lips and/or integuments.

This application claims benefit of U.S. Provisional Application No. 60/642,979, filed Jan. 12, 2005, the contents of which are incorporated herein by reference. This application also claims benefit of priority under 35 U.S.C. § 119 to French Patent Application No. 04 53263, filed Dec. 30, 2004, the contents of which are also incorporated herein by reference.

The present disclosure relates to cosmetic compositions comprising at least one alkoxylated ester. In one aspect, the cosmetic compositions may be chosen from cosmetic makeup and care compositions for human facial skin, body skin, scalp, lips, and integuments, such as, for example, hair, eyelashes, eyebrows, and nails.

The compositions disclosed herein, may, in certain embodiments, for example, constitute a makeup product (e.g., for the lips, body, or integuments) that may have care properties. The compositions disclosed herein may, for instance, be in the form of lipstick, lip gloss, makeup rouge, eye shadow, tattoo product, mascara, eyeliner, nail varnish, hair-coloring product, hair care product, or a product for artificially tanning the skin.

Certain alkoxylated esters have already been used in cosmetic compositions.

For example, U.S. Patent Application Publication No. 2002/0192249 describes cosmetic compositions comprising an ester of a monocarboxylic acid containing from 4 to 24 carbon atoms, and of an alcohol comprising a polypropoxyl group and an alkyl chain containing from 2 to 24 carbon atoms.

The preparations may also include mineral oil or liquid paraffin in addition to the alkoxylated ester. The document also describes an anhydrous composition containing this ester and a film-forming agent, and a composition containing PPG-3 myristyl ether neoheptanoate. Moreover, the document teaches lipstick compositions containing this polypropoxylated ester in combination with hydrogenated polyisobutene; cream foundation compositions as an emulsion containing this ester; and sunscreen compositions as an emulsion containing hexyl laurate, octyl palmitate and cetyl palmitate in combination with this ester.

U.S. Pat. No. 5,693,361 discloses cosmetic compositions containing an alkoxylated fatty ester obtained from a dicarboxylic acid containing from 2 to 22 carbon atoms, for instance maleic acid, and a stoichiometric excess of at least one polyalkoxylated fatty alcohol comprising an alkyl chain containing from 14 to 22 carbon atoms and a polyalkoxyl group. The preparations may comprise mineral oil or liquid paraffin as second emollient. The document also describes an anhydrous composition containing this ester and a film-forming agent. The alkoxylated ester can be, for example, di-PPG-3 myristyl maleate.

U.S. Pat. No. 6,476,254 discloses cosmetic compositions containing an ester of a dicarboxylic acid containing from 4 to 12 carbon atoms and a polyalkoxylated fatty alcohol in which the non-alkoxylated portion contains from 8 to 36 carbon atoms. The ester may be di-PPG-3 myristyl adipate. The composition may be anhydrous. It may contain mineral oil or liquid paraffin.

International Patent Application Publication No. WO 2003/013 439 relates to an ester of a C₃-C₂₁ dicarboxylic acid (or of a C₄-C₂₂, for instance, C₃ to C₉) aliphatic tricarboxylic acid and of a polyalkoxylated fatty alcohol comprising a C₆-C₃₀, such as a C₁₈-C₂₂ alkyl radical. The document describes a cosmetic composition that may contain petroleum jelly, mineral oil, esters derived from aliphatic carboxylic acids and aliphatic alcohols containing from 18 to 40 carbon atoms, film-forming agents, and fatty alcohols such as cetyl alcohol.

U.S. Pat. Nos. 5,302,377, 5,455,025 and 5,597,555 disclose cosmetic compositions containing an alkoxylated fatty ester of a tricarboxylic acid, such as citric acid, with a stoichiometric excess of at least one polyalkoxylated fatty alcohol with emollient properties, for topical preparations. The preparations contain a mineral oil as second emollient. The documents also describe the combination of an alkoxylated fatty ester of a tricarboxylic acid with a film-forming agent. The ester can be, for example, tri-PPG-3 myristyl citrate.

International Patent Application Publication No. WO 2004/052076 describes cosmetic compositions containing mixed esters of polyalkoxylated alcohols and of monohydric alcohols with polycarboxylic acids, such as dicarboxylic acids. These compositions may contain a second emollient such as mineral oil or petroleum jelly. The mixed esters described may be formulated in combination with a film-forming compound.

The inventors have discovered, surprisingly, that the combination of at least one alkoxylated ester and of at least one apolar oil can allow the production of formulations whose gloss is high and whose comfort is equivalent to that of a standard formulation.

Thus the present disclosure relates to a cosmetic composition comprising at least one alkoxylated ester derived from at least one alkoxylated alcohol and at least one carboxylic acid; and at least one apolar oil. In one embodiment, the composition can form a deposit with a mean gloss measured at 60° that is greater than or equal to 45 out of 100.

The present disclosure also relates to a cosmetic process producing a film of cosmetic composition gloss and comfort properties, the process comprising introducing into the composition at least one ester derived from at least one alkoxylated alcohol and at least one carboxylic acid; and at least one apolar oil.

The present disclosure still further relates to the cosmetic use of a composition as described above, and the cosmetic use of a combination of at least one ester derived from at least one alkoxylated alcohol and at least one carboxylic acid; and at least one apolar oil, to obtain a cosmetic composition that has gloss and comfort properties.

Also disclosed herein is a care and/or makeup process for keratin materials, comprising applying to the keratin materials a composition comprising at least one ester derived from at least one alkoxylated alcohol and at least one carboxylic acid; and at least one apolar oil, wherein when the composition forms a deposit, the deposit has a mean gloss measured at 60° that is greater than or equal to 45.

In the text hereinbelow, and unless otherwise indicated, the limits of a range of values are understood as forming part of that range.

As used herein, the term “alkoxylated alcohol” is understood to mean a hydrocarbon-based compound comprising at least one —OH functional group, for example one —OH functional group, and at least one group of Formula (I):

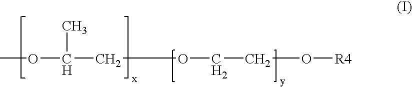

wherein x and y are, independently of each other, chosen from integers ranging from 0 to 40, and the sum of x and y ranges from 1 to 80,

and R₄ is chosen from aliphatic and aromatic, saturated and unsaturated, substituted and unsubstituted hydrocarbon-based groups comprising from 1 to 36 carbon atoms (such as, e.g., from 4 to 36 carbon atoms).

The alkoxylated alcohol can be, in certain embodiments, a polyalkoxylated alcohol wherein the group of Formula (I) is such that x and y are, independently from each other, chosen from integers ranging from 0 to 40, and the sum of x and y ranges from 2 to 80 inclusive. In one embodiment, x and y are, independently of each other, chosen from integers ranging from 0 to 30, and the sum of x and y ranges from 2 to 30.

In the embodiment depicted in Formula (I), all ethoxy units are depicted in a first group and all propoxy units are depicted in a second group. In general, however, the ethoxy and propoxy units may be placed in any order such as, e.g., randomly, in blocks, and in the form of alternating units. By way of non-limiting example, the ethoxy units (E) and the propoxy units (P) of the alkoxylated alcohol may be arranged as depicted in the following non-limiting examples: EEEP, EEPE, EPEE, PEEE, EEEPEPPPE, PEPPPEEEEPE, and in similar arrangements.

Alkoxylated Ester

Disclosed herein is a composition comprising at least one ester of an alkoxylated alcohol and of a carboxylic acid (referred to hereinbelow as an alkoxylated ester), which may be chosen from:
the esters obtained by reacting at least one monocarboxylic acid with at least one alkoxylated alcohol;
the polyesters obtained by reacting at least one polycarboxylic acid with a stoichiometric excess of at least one alkoxylated alcohol relative to the number of acid functional groups in the acid;
polyesters in which only one ester functional group is obtained by reacting an acid function of at least one polycarboxylic acid with at least one alkoxylated alcohol;
polyesters comprising at least one ester functional group obtained by reacting an acid functional group of at least one polycarboxylic acid with at least one alkoxylated alcohol, and at least one ester functional group obtained by reacting another acid functional group of the polycarboxylic acid with a fatty alcohol;
and mixtures thereof.
   Alkoxylated Ester of Monocarboxylic Acid

The at least one alkoxylated ester can be chosen from esters of monocarboxylic acids and of alkoxylated, such as, polyalkoxylated, fatty alcohols. In certain embodiments, the alkoxylated ester is chosen from the esters prepared by reacting an aliphatic or aromatic monocarboxylic acid with a stoichiometric excess of a polyalkoxylated fatty alcohol, for example a polypropoxylated alcohol.

The alkoxylated ester of a monocarboxylic acid may, for example, be chosen from the polypropoxylated monoesters of Formula (II): 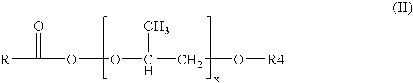
wherein x is an integer ranging from 2 to 40. For example, in certain embodiments x may range from 3 to 30, or from 3 to 10.

R₄ is chosen from saturated and unsaturated, substituted and unsubstituted, aliphatic hydrocarbon-based groups comprising from 1 to 36 carbon atoms (such as, e.g., from 3 to 24 carbon atoms, or from 4 to 24 carbon atoms), and RCOO is chosen from aliphatic and aromatic monocarboxylic acids RCOOH.

RCOO may be chosen from, for example:

monocarboxylic acid residues, such as, e.g., acids of formula (R₂R₃R₄C)COO in which R₂, R₃ and R₄ are chosen, independently from each other, from methyl, ethyl, propyl and isopropyl groups; and

aromatic acid residues comprising a benzenyl ring optionally substituted with, for example, a group chosen from —OH, —NH₂, methyl, and ethyl groups.

In some embodiments, the aliphatic monocarboxylic acids suitable for preparing the alkoxylated ester may comprise from 4 to 24 carbon atoms, such as, e.g., from 4 to 18 carbon atoms. Non-limiting examples of aliphatic monocarboxylic acids include 2-ethylhexanoic acid, caproic acid, neopentanoic acid, isostearic acid, neoheptanoic acid and oleic acid.

Non-limiting examples of aromatic monocarboxylic acids include benzoic acid and p-aminobenzoic acid.

The alkoxylated esters may be prepared from alkoxylated alcohols chosen from, saturated and unsaturated, substituted and unsubstituted, aliphatic and aromatic, and straight chain and branched chain alcohols. The alcohols may comprise, for example, from 6 to 24 carbon atoms, such as from 12 to 14 carbon atoms.

As used herein, the term “fatty alcohol” is understood to mean an aliphatic alcohol comprising at least three carbon atoms. For example, the fatty alcohol can comprise carbon, hydrogen and oxygen atoms. It may be saturated or may comprise at least one carbon-carbon double bond.

A fatty alcohol may be, for example, an alcohol obtained by hydrolysis of fats or of plant or animal oils.

The at least one ester derived from a monocarboxylic acid and a polypropoxylated fatty alcohol can be chosen, for example, from PPG-3 myristyl ether neoheptanoate (sold under the reference Trivasperse), PPG-4 butyloctyl ether ethylhexanoate, and mixtures thereof.

The at least one ester may be prepared according to the teaching of U.S. Patent Application Publication No. 2002/0192249, the contents of which are incorporated herein by reference.

Alkoxylated Mixed Polyesters

The at least one alkoxylated ester may also be chosen, in some embodiments, from mixed esters derived from at least one alkoxylated alcohol, at least one monohydric alcohol, and at least one polycarboxylic acid, such as, e.g., a polycarboxylic acid chosen from dicarboxylic acids.

The at least one alkoxylated ester is, in one embodiment, chosen from mixed esters of a polyalkoxylated fatty alcohol and of a monohydric fatty alcohol with dicarboxylic fatty acids.

The term “mixed ester,” as used herein, is understood to mean an ester obtained by reacting a polycarboxylic acid with at least two different alcohols.

The term “fatty acid,” as used herein, is understood to mean an aliphatic carboxylic acid comprising at least three carbon atoms. In one embodiment, for example, the fatty acid can comprise carbon, hydrogen and oxygen atoms. It may be saturated or may comprise at least one carbon-carbon double bond.

A fatty acid may be, for example, a carboxylic acid obtained by hydrolysis of fats or of plant or animal oils.

The at least one mixed ester of alkoxylated alcohol may be chosen, in certain embodiments, from compounds of Formula (III): 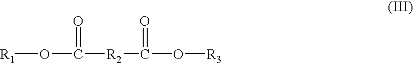
wherein R₁ is chosen from the compounds of Formula (IV): 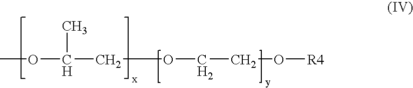
wherein:

R₄ is chosen from saturated and unsaturated, substituted and unsubstituted, aliphatic groups comprising from 4 to 24 carbon atoms;

x is an integer ranging from 3 to 30; y is an integer ranging from 3 to 30;

R₂ is chosen from saturated and unsaturated, substituted and unsubstituted, aliphatic groups comprising from 4 to 40 carbon atoms; and

R₃ is chosen from saturated and unsaturated, straight-chain and branched-chain aliphatic groups comprising from 4 to 32 carbon atoms (such as, e.g., from 12 to 24 carbon atoms).

Non-limiting examples of compounds corresponding to Formula (III) include:

octyldodecyl PPG-3 myristyl ether dimer dilinoleate (sold under the reference Liquiwax polyEFA by the company Arch Chemical), of Formula (V):

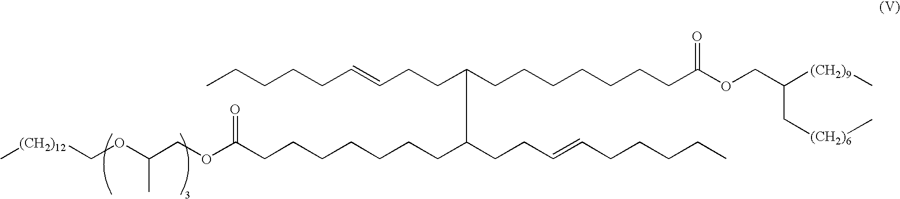

stearyl PPG-3 myristyl ether dimer dilinoleate (sold under the reference Liquiwax polylPL by the company Arch Chemical), and

isostearyl PPG-4 butyloctyl ether dimer dilinoleate.

The at least one mixed ester may be prepared by reacting an alkoxylated fatty alcohol and a monohydric fatty alcohol with at least one dicarboxylic fatty acid.

The alkoxylated fatty alcohol may be chosen, for example, from propoxylated fatty alcohols having a carbon chain length ranging from 4 to 24 carbon atoms, and a degree of propoxylation ranging from 3 to 30 (such as, e.g., ranging from 3 to 15) propylene oxide units. In one embodiment, the propoxylated fatty alcohol is chosen from propoxylated myristyl alcohol and propoxylated butyloctanol.

The dicarboxylic acid comprises at least two carboxylic groups per molecule. For example the dicarboxylic acid can be chosen from those of formula (VI):
HOOC—(CH₂)ₙ—COOH (VI)
wherein n is an integer ranging from 1 to 16 (such as, e.g., an integer ranging from 3 to 16).

Non-limiting examples of dicarboxylic acids that may be employed include, malonic acid, succinic acid, glutaric acid, adipic acid, pimelic acid, suberic acid, azelaic acid, sebacic acid, 1,9-nonamethylenedicarboxylic acid, 1,10-decamethylenedicarboxylic acid, 1,11-undecamethylenedicarboxylic acid, 1,12-dodecamethylenedicarboxylic acid, 1,13-tridecamethylenedicarboxylic acid, 1,14-tetradecamethylenedicarboxylic acid, 1,15-pentadecamethylenedicarboxylic acid, 1,16-hexadecamethylenedicarboxylic acid, and mixtures thereof.

The at least one dicarboxylic acid may also be, in one embodiment, a diacid dimer. The term “diacid dimer,” as used herein, is understood to mean a diacid obtained via an intermolecular polymerization (such as, e.g., a dimerization) reaction of at least one unsaturated monocarboxylic acid.

The diacid dimer may be derived, for example, from the dimerization of an unsaturated fatty acid. For example, a diacid dimer may be derived from the dimerization of a C₈ to C₃₄, for instance C₁₂ to C₂₂, such as C₁₆ to C₂₀, or C₁₈ unsaturated fatty acid.

Non-limiting examples of unsaturated fatty acids include, undecenoic acid, linderic acid, myristoleic acid, palmitoleic acid, oleic acid, linoleic acid, elaidinic acid, gadolenoic acid, eicosapentenoic acid, docosahexenoic acid, erucic acid, brassidic acid, arachidonic acid, and mixtures thereof.

In one embodiment of the present disclosure, the diacid dimer can be the diacid dimer from which the diol dimer to be esterified is also derived.

In another embodiment, the diacid dimer can be the diacid dimer obtained by dimerization of linoleic acid, optionally followed by hydrogenation of the carbon-carbon bonds. The diacid dimer may be in saturated form, i.e., it may comprise no carbon-carbon double bonds. According to another embodiment, the possible carbon-carbon double bonds in the diacid dimer are all or partly hydrogenated, after an esterification reaction of the diacid dimer with the diol dimer.

According to still another embodiment, the diacid dimer is a commercially available dicarboxylic acid comprising about 36 carbon atoms. This product may also comprise a trimeric acid and a monomeric acid, in proportions that depend on the degree of purity of the product. Products wherein the content of diacid dimer is greater than 70%, and products wherein the content of diacid dimer has been adjusted to greater than or equal to 90%, are conventionally found commercially.

Diacid dimers, such as, e.g., dilinoleic diacids stabilized towards oxidation by hydrogenation of double bonds remaining after the dimerization reaction may also be purchased commercially.

According to the present disclosure, any currently commercially available diacid dimer may be used.

The monohydric fatty alcohol may have a carbon chain length ranging from 12 to 24 carbons. In one embodiment, the monohydric fatty alcohol is chosen from octyldodecanol and isostearyl alcohol.

Examples of preparation of the esters described above are given in International Patent Application Publication No. WO 2004/052076, the content of which is incorporated herein by reference.

Alkoxylated Polyesters

The at least one alkoxylated ester may also be obtained by esterification of a polycarboxylic acid with at least two alkoxylated alcohols, which may be identical or different, so as to form an ester.

For example, the at least one ester may be chosen, in some embodiments, from the esters of Formula (VII): 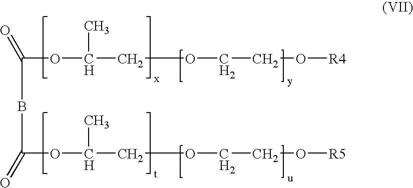
wherein:

—OOC—B—COO— is chosen from saturated and unsaturated, substituted and unsubstituted, dicarboxylic acid residues, as described above, such as dicarboxylic acid residues comprising from 2 to 40 carbon atoms, wherein B is a linking group containing up to 38 carbon atoms or a bond,

x and y are, independently from each other, chosen from integers ranging from 0 to 40, and the sum of x and y ranges from 1 to 80 (such as, e.g., from 2 to 80),

t and u are, independently from each other, chosen from integers ranging from 0 to 40, and the sum of t and u ranges from 1 to 80 (such as, e.g., from 2 to 80),

R₄ and R₅ are, independently from each other, chosen from aliphatic and aromatic, saturated and unsaturated, substituted and unsubstituted hydrocarbon-based units comprising from 4 to 36 carbon atoms.

According to one embodiment, R₄ and R₅, which may be identical or different comprise from 10 to 22 carbon atoms, and may be chosen from saturated and unsaturated, substituted and unsubstituted hydrocarbon-based units.

According to another embodiment, y ranges from 1 to 40 and x ranges from 0 to 30, wherein if x is equal to 0, y is at least equal to 2, and further wherein y is greater than x.

According to one embodiment, u ranges from 1 to 40 and t ranges from 0 to 30, wherein if t is equal to 0, u is at least equal to 2, and further wherein u is greater than t.

In another embodiment, the dicarboxylic acid is chosen from aliphatic dicarboxylic acids comprising from 2 to 36 carbon atoms, such as, e.g., from 3 to 8 carbon atoms. Non-limiting examples of suitable aliphatic dicarboxylic acids include adipic acid, sebacic acid, malonic acid, succinic acid, and maleic acid.

In yet another embodiment, the dicarboxylic acid is chosen from aromatic dicarboxylic acids comprising from 8 to 36 carbon atoms. For example, the aromatic dicarboxylic acid may be chosen from aromatic dicarboxylic acids comprising from 8 to 12 carbon atoms. Anon-limiting example of a suitable aromatic dicarboxylic acid is phthalic acid, such as, e.g., 1,2-phthalic acid, which has the lowest melting point of the phthalic acid isomers.

In one embodiment, x and y may each be equal to 15 with the proviso that the total of x and y does not exceed 25. In another embodiment, u and t may each be equal to 15 with the proviso that the total of u and t does not exceed 25.

In yet another embodiment, y or u is greater than or equal to 1, and x or t is greater than or equal to 0. The number of ethoxy units may, in some embodiments, be greater than the number of propoxy units.

The alkoxylated esters may be prepared according to the teaching of International Patent Application Publication No. WO 00/19972, the content of which is incorporated herein by reference.

The dicarboxylic acid diesters of the present disclosure may comprise, for example, unsubstituted saturated aliphatic groups. In certain embodiments, the unsubstituted saturated aliphatic groups can comprise from 14 to 18 carbon atoms or from 14 to 16 carbon atoms. In other embodiments, the unsubstituted saturated aliphatic group is the myristyl fatty group, comprising 14 carbon atoms.

In one embodiment, R₄ and R₅ are each a myristyl group, y and u are each equal to zero, and each of x and t is an integer chosen, independently from each other, from integers ranging from 2 to 40. One example is the product sold under the reference Cromollient DP3A wherein, in Formula (VII), R₄ and R₅ are each a myristyl group, —OOC—B—COO— is an adipate, y and u are both equal to 0, and x and t are both equal to 3.

The at least one alkoxylated ester can be present in the composition in an amount ranging from 1% to 99% by weight (such as from 2% to 60% by weight, from 5% to 40% by weight, or from 10% to 35% by weight) relative to the total weight of the composition.

Apolar Oil

The composition according to the present disclosure comprises at least one apolar oil. As used herein, the term “apolar oil” is understood to mean an oil whose solubility parameter at 25° C., δₐ, is equal to 0 (J/cm³)^{1/2}.

The definition and calculation of the solubility parameters in the Hansen three-dimensional solubility space are described in the article by C. M. Hansen: “The three dimensional solubility parameters” J. Paint Technol. 39, 105 (1967).

According to this Hansen space:

δ_{D} represents the London dispersion forces derived from the formation of dipoles induced during molecular impacts;

δₚ represents the Debye interaction forces between permanent dipoles and also the Keesom interaction forces between induced dipoles and permanent dipoles;

δₕ represents the specific interaction forces (such as hydrogen bonding, acid/base, donor/acceptor, etc.);

δₐ is determined by the equation: δₐ=(δₚ²+δₕ²)^{1/2}.

The parameters δₚ, δₕ, δ_{D} and δₐ are expressed in (J/cm³)^{1/2}.

The at least one apolar oil may be hydrocarbon-based.

As used herein, the term “hydrocarbon-based oil” is understood to mean an oil comprising, or even consisting of, carbon and hydrogen atoms, and possibly oxygen and nitrogen atoms, and containing no silicon or fluorine atoms. It may comprise alcohol, ester, ether, carboxylic acid, amine and/or amide groups. In one embodiment, the at least one apolar hydrocarbon-based oil is free of heteroatoms. The term “heteroatom,” as used herein, is understood to mean an atom other than carbon or hydrogen.

The at least one apolar oil can be, in some embodiments, non-volatile.

The term “non-volatile oil,” as used herein, is understood to mean any oil having a non-zero vapor pressure at room temperature and atmospheric pressure (such as, e.g., a vapor pressure of less than 0.02 mm Hg or less than or equal to 10⁻³ mm Hg.

The at least one apolar oil can be present in the composition in an amount ranging from 1% to 80% by weight, relative to the total weight of the composition, such as, e.g., from 3% to 50% or from 5% to 30% by weight, relative to the total weight of the composition.

For example, the at least one apolar oil may be present in an amount ranging from 10% to 40% by weight relative to the total weight of the composition, such as, e.g., from 15% to 30% by weight relative to the total weight of the composition.

In one embodiment, the at least one apolar oil is a non-volatile hydrocarbon-based apolar oil chosen from linear and branched saturated alkanes.

The at least one apolar oil may be chosen from oils with a molecular mass ranging from 300 g/mol to 900 g/mol (such as, e.g, oils with a molecular mass ranging from 350 g/mol to 800 g/mol).

Non-limiting examples of apolar oils include:

hydrocarbon-based oils, (for instance, squalene; linear and branched hydrocarbons such as liquid paraffin, liquid petroleum jelly and naphthalene oil, hydrogenated or partially hydrogenated polyisobutene, isoeicosane, squalane, decene/butene copolymers, polybutene/polyisobutene copolymers (such as, e.g., Indopol L-14), and polydecenes such as Puresyn 10, and mixtures thereof);

polydimethylsiloxanes (PDMS), optionally comprising a C₃-C₄₀ alkyl or C₃-C₄₀ alkoxy chain, or a phenyl radical (for instance, polydimethylsiloxanes comprising phenylated radicals may be chosen from phenyl trimethicones);

optionally fluorinated polyalkylmethylsiloxanes, for example polymethyltrifluoropropyldimethylsiloxanes;

polyalkylmethylsiloxanes substituted with at least one functional group such as, e.g., hydroxyl, thiol and amine groups;

polysiloxanes modified with fatty acids, fatty alcohols or polyoxyalkylenes; and

mixtures thereof.

The composition of the present disclosure may comprise several apolar oils. According to one embodiment, the mass ratio between the at least one alkoxylated alcohol ester and the at least one apolar oil ranges from 20/80 to 80/20. For example, the mass ratio may range from 30/70 to 70/30, or from 40/60 to 60/40. In certain embodiments, the mass ratio is about 50/50.

Gloss

According to certain embodiments, the composition can form a deposit having a mean gloss, measured at 60°, of greater than or equal to 45. The at least one apolar oil is, in some embodiments, such that when it is present in sufficient amount, the composition can form a deposit having a mean gloss, measured at 60°, of greater than or equal to 50.

As used herein, the term “mean gloss” is understood to mean the gloss as measured using a glossmeter, in a conventional manner via the following method:

A coat 25 μm thick of the composition is spread onto a Byk Gardner brand contrast card of reference Prüfkarten, Art. 2853, premounted onto a 1 mm glass plate, using an automatic spreader (Bar coater, Sheen). The coat covers at least the black background of the card. When the composition is solid, it is melted, if necessary, on the card after having been spread, so that it covers the black background. Once the composition has been spread, the measurement of the gloss, known as the mean gloss T0h, is measured at 600 on the black background using a Byk Gardner brand glossmeter of reference microTri-Gloss. Thus, four contrast cards are prepared to measure the mean gloss of the composition, and the mean of the four measurements is determined. In order for the measurement to be correct, the standard deviation must be less than or equal to 3%.

The contrast card is then left for 5 hours on a plate thermostatically maintained at a temperature of 30° C. After 5 hours, the contrast card is removed from the thermostatically regulated plate so that it returns to the temperature of the room, and measurement of the mean gloss, known as the mean gloss T5h, is again performed as previously.

In one embodiment, the mean gloss of the composition T0h, once spread onto a support, measured at 60° C., is greater than or equal to 45 (such as, e.g., greater than or equal to 50, greater than or equal to 60, greater than or equal to 65, greater than or equal to 70, or greater than or equal to 75) out of 100.

In another embodiment, the mean gloss of the composition T5h, once spread onto a support, measured at 60° C., is greater than or equal to 35 (such as, e.g., greater than or equal to 40, greater than or equal to 45, greater than or equal to 50, greater than or equal to 55, greater than or equal to 60, greater than or equal to 65, greater than or equal to 70, or greater than or equal to 75) out of 100.

According to one embodiment, the composition can have good staying power of the gloss over time. In particular, the mean loss of gloss over time is less than or equal to 25%. For example, the mean loss of gloss over time may be less than or equal to 20%, less than or equal to 15%, less than or equal to 10%, or less than or equal to 5%.

The loss of gloss can be expressed, for example, as the ratio (T0h−T5h)/T0h.

According to one embodiment, the mean gloss T0h is greater than or equal to 70 out of 100, and the mean gloss T5h is greater than or equal to 70 out of 100. According to another embodiment, the mean gloss T0h is greater than or equal to 75 out of 100, and the mean gloss T5h is greater than or equal to 75 out of 100.

Pasty Substance

The composition may also comprise at least one pasty compound.

The composition can be, in certain embodiments, free of lanolin or of lanolin derivatives.

Thus, one embodiment of the present disclosure is a cosmetic composition comprising at least one ester of an alkoxylated alcohol and of a carboxylic acid and at least one apolar oil, wherein the composition can form a deposit having a mean gloss measured at 60° C. of greater than or equal to 30 out of 100, and wherein the composition is free of lanolin or derivatives thereof. In this embodiment, the gloss may be measured according to the method described above. For example, the gloss may be greater than or equal to 35 out of 100, or greater than or equal to 40 out of 100.

Non-limiting examples of conventionally used lanolin derivatives include, e.g., liquid lanolin, reduced lanolin, adsorption-purified lanolin, acetylated lanolin, oxypropylenated (5 PO) lanolin wax, liquid lanolin acetate, hydroxylanolin, polyoxyethylene-lanolin, lanolin fatty acid, hard lanolin fatty acid, cholesteryl esters of lanolin fatty acid, lanolin alcohol, lanolic alcohol acetate, isopropyl lanolate, and the like.

Lanolins can have the drawback of being sensitive to heat and to ultraviolet radiation. They have a tendency to become oxidized and that can evolve into an unpleasant odor, and their strong yellow color prevents them from being used in unpigmented care bases and colorless bases, which limits their use in cosmetic compositions.

The Inventors have found that the alkoxylated esters described above are good substitutes for lanolin and derivatives thereof.

For the purposes of the present disclosure, the term “pasty substance” is understood to mean a lipophilic fatty compound, with a reversible solid/liquid change of state, wherein the lipophilic fatty compound comprises, at a temperature of 23° C., a liquid fraction and a solid fraction. The term “pasty substance” also refers to polyvinyl laurate.

The at least one pasty compound may be chosen from, for example:

lanolin and its derivatives;

polymeric and non-polymeric fluoro compounds;

polymeric and non-polymeric silicone compounds;

vinyl polymers, such as, e.g.:
olefin homopolymers;
olefin copolymers;
hydrogenated diene homopolymers and copolymers;
homopolymeric and copolymeric, linear and branched oligomers of alkyl (meth)acrylates (such as, e.g., alkyl acrylates comprising a C₈-C₃₀ alkyl group);
homopolymeric and copolymeric, linear and branched oligomers of alkyl (meth)acrylates (such as, e.g., alkyl methacrylates comprising a C₈-C₃₀ alkyl group);
homopolymeric and copolymeric oligomers of vinyl esters comprising C₈-C₃₀ alkyl groups;
homopolymeric and copolymeric oligomers of vinyl ethers comprising C₈-C₃₀ alkyl groups;
liposoluble polyethers resulting from polyetherification between at least one C₂-C₁₀₀ diol (such as, e.g., at least one C₂-C₅₀ diol);
esters,
and mixtures thereof.

In some embodiments, the at least one liposoluble polyether can be chosen from copolymers of ethylene oxide and/or of propylene oxide with C₆-C₃₀ long-chain alkylene oxides. In certain embodiments, the weight ratio of the ethylene oxide and/or of the propylene oxide to the alkylene oxides in the copolymer ranges from 5:95 to 70:30. In these embodiments, the liposoluble polyethers may be copolymers such that the long-chain alkylene oxides are arranged in blocks having a mean molecular weight ranging from 1,000 to 10,000, for example a polyoxyethylene/polydodecyl glycol block copolymer such as, e.g., the ethers of dodecanediol (22 mol) and of polyethylene glycol (45 EO) sold under the brand name Elfacos ST9 by Akzo Nobel.

The at least one pasty ester may be chosen from, e.g.:
esters of an oligomeric glycerol (such as, e.g., diglycerol esters, including condensates of adipic acid and of glycerol), for which some of the hydroxyl groups of the glycerols have reacted with a mixture of fatty acids (such as, e.g., stearic acid, capric acid, stearic acid and isostearic acid, and 12-hydroxystearic acid, including those sold under the brand name Softisan 649 by the company Sasol);
the arachidyl propionate sold under the brand name Waxenol 801 by Alzo,
phytosterol esters;
non-crosslinked polyesters resulting from the polycondensation between a linear or branched C₄-C₅₀ dicarboxylic or polycarboxylic acid and a C₂-C₅₀ diol or polyol, other than the polyester described above;
aliphatic esters of an ester derived from the esterification of an ester of an aliphatic hydroxycarboxylic acid with an aliphatic monocarboxylic acid; and mixtures thereof, for example
   the ester derived from the esterification reaction of hydrogenated castor oil with isostearic acid in proportions of 1 to 1 (1/1) or hydrogenated castor oil monoisostearate;
   the ester derived from the esterification reaction of hydrogenated castor oil with isostearic acid in proportions of 1 to 2 (1/2) or hydrogenated castor oil diisostearate;
   the ester derived from the esterification reaction of hydrogenated castor oil with isostearic acid in proportions of 1 to 3 (1/3) or hydrogenated castor oil triisostearate;
   and mixtures thereof.

Among the pasty compounds of plant origin that may be used, non-limiting mention may be made of a mixture of soybean sterols and of oxyethylenated (5 EO) oxypropylenated (5 PO) pentaerythritol, sold under the reference Lanolide by the company Vevy.

The at least one pasty compound, when present, can be present in an amount ranging from 1% to 99%, from 1% to 60%, from 2% to 30%, or from 5% to 15% by weight, relative to the total weight of the composition.

Dye

The compositions of the present disclosure may also further comprise at least one dyestuff, which may be chosen from dyes, pigments and nacres, and mixtures thereof. The at least one dyestuff may be present in an amount ranging from 0.001% to 98%, from 0.5% to 85%, or from 1% to 60% by weight, relative to the total weight of the composition.

The at least one dye can be, for example, a liposoluble dye, although water-soluble dyes may also be used. The at least one liposoluble dye may be chosen from, for example, Sudan red, D & C Red 17, D & C Green 6, β-carotene, soybean oil, Sudan brown, D & C Yellow 11, D & C Violet 2, D & C Orange 5, quinoline yellow and annatto. The at least one liposoluble dye may be present in an amount ranging from 0% to 20% by weight, relative to the total weight of the composition, such as, e.g., from 0.1% to 6% of the weight of the composition. The at least one water-soluble dye may be chosen, for example, from beetroot juice and methylene blue.

For a composition in paste or cast form (such as, e.g., a lipstick or a body makeup product), the at least one dyestuff may be present in an amount ranging from 0.5% to 50% (such as, e.g., from 2% to 40%, or from 5% to 30%) by weight, relative to the total weight of the composition.

The term “pigments,” as used herein, is understood to mean white and colored, mineral and organic particles that are insoluble in the at least one oil of the composition, which are intended to color and/or opacify the composition. The term “fillers,” as used herein, is understood to mean colorless and white, mineral and synthetic, lamellar and non-lamellar particles. The term “nacres,” as used herein, is understood to mean iridescent particles, such as those produced by certain mollusks in their shell. Alternatively, the at least one nacre may be chosen from synthetic nacres. These fillers and nacres can serve, in some embodiments, to modify the texture of the composition.

The at least one pigment, when present, may be present in the composition in an amount ranging from 0.05% to 30% (such as, e.g., from 2% to 20%) by weight, relative to the weight of the final composition. Mineral pigments may be used in the present disclosure, including, by way of non-limiting example, titanium oxide, zirconium oxide, cerium oxide, zinc oxide, iron oxide, chromium oxide, and ferric blue. Among the organic pigments that may be used in the present disclosure, non-limiting mention may be made of carbon black, and barium, strontium, calcium (D & C Red No. 7), and aluminium lakes.

The at least one nacre, when present, may be present in the composition in an amount ranging from 0.001% to 20% (such as, e.g., from 1% to 15%) by weight, relative to the total weight of the composition. The at least one nacre may be chosen from, for example, mica coated with titanium oxide, with iron oxide, with natural pigment, or with bismuth oxychloride, such as, e.g., colored titanium mica.

The composition, in certain embodiments, may comprise goniochromatic pigments, for example multilayer interference pigments, and/or reflective pigments. These two types of pigment are described in French Patent Application No. FR 02/09246, which is incorporated herein by reference.

Fillers

The composition may further comprise at least one filler, which may be present in an amount ranging from 0.001% to 35% (such as, e.g., from 0.5% to 15%) by weight, relative to the total weight of the composition.

The at least one filler may be chosen from, for example:
talc, mica, kaolin and starch
Nylon® powders (such as, e.g., Orgasol)
polyethylene powders
polytetrafluoroethylene (Teflon®) powders
boron nitride
copolymer microspheres (such as, e.g., Expancel® (Nobel Industrie))
Polytrap® 603 (Dow Corning)
Polypore® L 200 (Chemdal Corporation)
silicone resin microbeads (such as, e.g., Tospearl® from Toshiba)
silica-based fillers (such as, e.g., Aerosil 200, Aerosil 300; Sunsphere L-31 and Sunsphere H-31 sold by Asahi Glass; Chemicelen sold by Asahi Chemical); composites of silica and of titanium dioxide (such as, e.g., the TSG series sold by Nippon Sheet Glass)
polyurethane powders (such as, e.g., powders of crosslinked polyurethane comprising a copolymer, the copolymer comprising trimethylol hexyllactone. In some embodiments, the copolymer may be a polymer of hexamethylene diisocyanate/trimethylol hexyllactone, such as the particles commercially available, for example, under the name Plastic Powder D-400® or Plastic Powder D-800® from the company Toshiki)
N-acylamino acids, which in certain embodiments comprise an acyl group comprising from 8 to 22 carbon atoms (such as, e.g., a 2-ethylhexanoyl, caproyl, lauroyl, myristoyl, palmitoyl, stearoyl or cocoyl group), for example lauroyllysine.

The filler may be, for example, a filler with a mean particle size of less than 100 μm, such as ranging from 1 μm 50 μm, for example from 4 μm to 20 μm.

The filler may be in any form, such as, e.g., essentially spherical or in the form of platelets.

Wax

The composition may also further comprise at least one wax. As used herein, the term “wax” is understood to mean a lipophilic fatty compound that is solid at room temperature (25° C.), with a reversible solid/liquid change of state, that has a melting point of greater than 30° C. and less than or equal to 200° C., a hardness of greater than 0.5 MPa, and an anisotropic crystal organization in the solid state. By bringing the wax to its melting point, it is possible to make it miscible with oils and to form a microscopically homogeneous mixture, and upon returning the temperature of the mixture to room temperature, recrystallization of the wax in the oils of the mixture can be obtained.

The waxes that may be used in the present disclosure are compounds that are solid at room temperature, which are intended to structure the composition in, for example, stick form; they may be hydrocarbon-based waxes, fluoro waxes and/or silicone waxes and may be of plant, mineral, animal and/or synthetic origin. For instance, they may have a melting point of greater than 40° C., such as, e.g., greater than 45° C.

Among waxes that may be used according to the present disclosure, non-limiting mention may be made of those generally used in cosmetics: they are generally of natural origin, for example beeswax, carnauba wax, candelilla wax, ouricoury wax, Japan wax, cork fiber wax, sugarcane wax, rice wax, montan wax, paraffin, lignite wax or microcrystalline wax, ceresin or ozokerite, and hydrogenated oils, for example jojoba oil; synthetic waxes, for example the polyethylene waxes derived from the polymerization or copolymerization of ethylene and Fischer-Tropsch waxes, or alternatively fatty acid esters, for example octacosanyl stearate, glycerides that are solid at 40° C. (such as glycerides that are solid at 45° C.), silicone waxes, for example alkyl or alkoxy dimethicones comprising an alkyl or alkoxy chain of 10 to 45 carbon atoms, and poly(di)methylsiloxane esters that are solid at 40° C., the ester chain of which comprises at least 10 carbon atoms; and mixtures thereof.

The compositions described herein may comprise at least one polyethylene wax with a weight-average molecular mass ranging from 300 to 700, such as, e.g., a weight-average molecular mass of 500 g/mol.

As a guide, the wax may be present in an amount ranging from 0.01% to 50%, such as, e.g., from 2% to 40%, or from 5% to 30% by weight, relative to the total weight of the composition.

Non-Volatile Oil

The composition may also further comprise at least one non-volatile oil other than the alkoxylated alcohol ester and other than the apolar oil described above. The at least one non-volatile oil may be chosen from:
hydrocarbon-based plant oils such as, liquid triglycerides of fatty acids comprising from 4 to 10 carbon atoms (such as, e.g., heptanoic acid triglyceride, octanoic acid triglyceride, and jojoba oil);
hydrocarbon-based esters of formula RCOOR′ in which RCOO is chosen from carboxylic acid residues comprising from 2 to 30 carbon atoms and R′ is chosen from hydrocarbon-based chains comprising from 1 to 30 carbon atoms such as, e.g., isononyl isononanoate, oleyl erucate or 2-octyldodecyl neopentanoate;
fatty alcohols comprising from 12 to 26 carbon atoms (such as, e.g., octyldodecanol, 2-butyloctanol, 2-hexyldecanol, 2-undecylpentadecanol and oleyl alcohol);
fluoro oils that are optionally partially hydrocarbon-based and/or silicone-based;
fatty acids comprising from 12 to 26 carbon atoms (such as, e.g., oleic acid); and
mixtures thereof.
   Non-Volatile Oil of High Molecular Mass

In one embodiment of the present disclosure, the composition comprises at least one non-volatile oil of high molecular mass. For example, the at least one non-volatile oil of high molecular mass may have a molecular mass ranging from 650 g/mol to 10,000 g/mol.

The composition according to the present disclosure, in certain embodiments, comprises at least one oil with a molar mass ranging from 650 g/mol to 10,000 g/mol (such as, ranging from 900 g/mol to 7,500 g/mol), in an amount ranging from 2% to 30% (e.g., from 5% to 25% or from 5% to 15%) by weight, relative to the total weight of the composition.

The at least one non-volatile oil of high molecular mass may be an apolar oil as described above, given that, according to one embodiment of the present disclosure, the at least one apolar oil of the composition has a molecular mass ranging from 300 g/mol to 900 g/mol.

Thus, the at least one oil with a molecular mass (i.e., molar mass or MM) ranging from 650 g/mol to 10,000 g/mol may be chosen from:

polybutylenes such as Indopol H-100 (MM=965 g/mol), Indopol H-300 (MM=1340 g/mol) and Indopol H-1500 (MM=2160 g/mol) sold or manufactured by the company Amoco,

hydrogenated polyisobutylenes such as Panalane H-300 E sold or manufactured by the company Amoco (M=1,340 g/mol), Viseal 20000 sold or manufactured by the company Synteal (MM=6,000 g/mol) and Rewopal PIB 1000 sold or manufactured by the company Witco (MM=1,000 g/mol),

polydecenes and hydrogenated polydecenes such as Puresyn 150 (MM=9,200 g/mol) sold by the company Mobil Chemicals,

vinylpyrrolidone copolymers such as the vinylpyrrolidone/1-hexadecene copolymer Antaron V-216 sold or manufactured by the company ISP (MM=7300 g/mol),

esters such as:
a) linear fatty acid esters with a total carbon number ranging from 35 to 70, for example pentaerythrityl tetrapelargonate (MM=697.05 g/mol),
b) hydroxylated esters such as polyglyceryl-2 triisostearate (MM=965.58 g/mol),
c) aromatic esters such as tridecyl trimellitate (MM=757.19 g/mol),
d) esters of branched C₂₄-C₂₈ fatty alcohols or fatty acids (such as those described in European Patent Application No. EP-A-0 955 039), including, for instance, triisoarachidyl citrate (MM=1,033.76 g/mol), pentaerythrityl tetraisononanoate (MM=697.05 g/mol), glyceryl triisostearate (MM=891.51 g/mol), glyceryl 2-tridecyl tetradecanoate (MM=1,143.98 g/mol), pentaerythrityl tetraisostearate (MM=1,202.02 g/mol), polyglyceryl-2 tetraisostearate (MM=1,232.04 g/mol), and pentaerythrityl 2-tetradecyl tetradecanoate (MM=1,538.66 g/mol),
e) diol dimer esters and polyesters (such as, e.g., esters of diol dimer and of fatty acid, and esters of diol dimer and of diacid).
   The esters of diol dimer and of monocarboxylic acid may be prepared from linear, branched, saturated, and unsaturated monocarboxylic acids comprising from 4 to 34 carbon atoms (for example, comprising from 10 to 32 carbon atoms). For example, the monocarboxylic acid may be chosen from fatty acids.
   The at least one ester of a diol dimer and of a dicarboxylic acid may be prepared from a diacid dimer derived, for example, from the dimerization of an unsaturated fatty acid (such as a C₈ to C₃₄, C₁₂ to C₂₂, or C₁₆ to C₂₀ unsaturated fatty acid, for example a C₁₈ fatty acid).
   In one embodiment, it is the diacid dimer from which the diol dimer to be esterified is also derived.
   The at least one diol dimer ester may be prepared from a diol dimer produced by catalytic hydrogenation of a diacid dimer as described above, such as, for example, hydrogenated dilinoleic diacid.
   Non-limiting examples of diol dimer esters include the esters of dilinoleic diacids and of dilinoleyl diol dimers sold by the company Nippon Fine Chemical under the trade names Lusplan DD-DA5® and DD-DA7®,
silicone oils such as, phenyl silicones, for example Belsil PDM 1000 from the company Wacker (MM=,000 g/mol),
oils of plant origin such as, e.g., sesame oil (820.6 g/mol), and
mixtures thereof.

The at least one non-volatile oil of high molecular mass may be present in an amount ranging from 0.001% to 90%, from 0.05% to 60%, or from 1% to 35% by weight, relative to the total weight of the composition.

Volatile Oil

The composition may further comprise at least one volatile oil.

As used herein, the term “volatile oil” is understood to mean an oil (or non-aqueous medium) capable of evaporating on contact with the skin in less than one hour at room temperature and atmospheric pressure. The at least one volatile oil may be a volatile cosmetic oil that is liquid at room temperature and atmospheric pressure. The at least one volatile oil may, in one embodiment, have a non-zero vapor pressure, such as a vapor pressure ranging from 0.13 Pa to 40,000 Pa (10⁻³ mm Hg to 300 mm Hg), ranging from 1.3 Pa to 13,000 Pa (0.01 mm Hg to 100 mm Hg), or ranging from 1.3 Pa to 1300 Pa (0.1 mm Hg to 10 mm Hg).

In one embodiment of the present disclosure, the at least one volatile oil may have a boiling point, measured at atmospheric pressure, ranging from 150° C. to 260° C. For example, the boiling point may range from 170° C. to 250° C.

As used herein, the term “hydrocarbon-based oil” is understood to mean an oil comprising, carbon and hydrogen atoms, and possibly oxygen and nitrogen atoms, and containing no silicon or fluorine atoms; it may comprise ester, ether, amine or amide groups.

As used herein, the term “silicone oil” is understood to mean an oil comprising at least one silicon atom. The silicone oil, in one embodiment, comprises Si—O groups.

As used herein, the term “fluoro oil” is understood to mean an oil comprising at least one fluorine atom.

The at least one volatile oil may be a silicone oil or a hydrocarbon-based oil.

The at least one volatile silicone oil may be chosen from silicone oils with a flash point ranging from 40° C. to 102° C. In one embodiment, the at least one volatile silicone oil is chosen from volatile silicone oils with a flash point ranging from 55° C. to 95° C. For example, the flash point may range from 65° C. to 95° C.

The at least one volatile silicone oil may be chosen from linear and cyclic silicone oils with a viscosity at room temperature of less than 8 cSt. For example, the at least one volatile silicone oil may comprise from 2 to 7 silicon atoms, and may optionally comprise at least one alkyl or alkoxy group comprising from 1 to 10 carbon atoms. The at least one volatile silicone oil may be chosen from, for instance, octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane and decamethyltetrasiloxane, and mixtures thereof.

The at least one volatile silicone oil may be chosen from the silicones described in the French Patent Application No. FR 03/04259 (published as FR 2 853 227).

The at least one volatile hydrocarbon-based oil may be chosen from hydrocarbon-based oils having a flash point ranging from 40° C. to 102° C., such as, e.g., hydrocarbon-based oils having a flash point ranging from 40° C. to 55° C., or ranging from 40° C. to 50° C.

The at least one volatile hydrocarbon-based oil may be chosen from volatile hydrocarbon-based oils comprising from 8 to 16 carbon atoms, and mixtures thereof, such as branched C₈-C₁₆ alkanes, for instance C₈-C₁₆ isoalkanes (also known as isoparaffins), isododecane, isodecane, and isohexadecane (such as, for example the oils sold under the trade names Isopar or Permethyl), and branched C₈-C₁₆ esters (for example isohexyl neopentanoate), and mixtures thereof. In one embodiment, the at least one volatile hydrocarbon-based oil is chosen from volatile hydrocarbon-based oils comprising from 8 to 16 carbon atoms (such as isododecane, isodecane and isohexadecane) and mixtures thereof. In one embodiment, the at least one volatile hydrocarbon-based oil is isododecane.

The at least one volatile oil may be present in an amount ranging, for example, from 5% to 97.5% (such as from 10% to 75% or from 20% to 50%) of the total weight of the composition.

The at least one volatile oil, when present, may be present in an amount ranging, for example, from 20% to 50% or from 30% to 40% by weight, relative to the weight of the composition. For example, the at least one volatile oil may be present in an amount of 35% by weight, relative to the weight of the composition.

Adjuvants

The composition of the present disclosure may also comprise at least one additional adjuvant chosen from those commonly used in cosmetics, such as water, antioxidants, film-forming polymers, preserving agents, neutralizers, plasticizers, lipophilic gelling agents, liquid non-aqueous compounds, aqueous-phase-gelling agents, dispersants, and cosmetic active agents. The at least one adjuvant, with the exception of water, may be present in the composition in an amount ranging from 0.0005% to 20%, or, for example, in an amount ranging from 0.001% to 10% by weight, relative to the total weight of the composition. Water may be present in an amount ranging from 0% to 70% (for example, ranging from 1% to 50% or from 1% to 10%) by weight, relative to the total weight of the composition,

Among the cosmetic active agents that may be used according to the present disclosure, non-limiting mention may be made of, for example, vitamins A, E, C, B₃, and F, provitamins (for example, D-panthenol, glycerol, calmatives (for example, x-bisabolol), aloe vera, allantoin, plant extracts, plant essential oils, protecting agents, restructuring agents (for example, ceramides), “refreshing” active agents (for example, menthol and its derivatives), emollients (for example, cocoa butter and dimethicone), moisturizers (for example, arginine PCA), anti-wrinkle active agents, essential fatty acids, and sunscreens.

Needless to say, a person skilled in the art will take care to select the optional additional additives and/or the amount thereof such that the beneficial properties of the composition according to the present disclosure are not, or are not substantially, adversely affected by the envisaged addition.

Galenical Forms

The compositions described herein have many applications for colored and uncolored cosmetic products. In certain embodiments, the compositions described herein may be used as lipsticks.

The compositions described herein may be, e.g., in the form of a solid, compacted or cast composition, such as a stick or a dish, or in pasty or liquid form. The compositions may be, in one embodiment, in a solid form, i.e., in a hard form (not flowing under its own weight) that may be cast or compacted, for example as a stick or a dish.

The compositions described herein may be in the form of a paste, solid, or cream. The compositions described herein may be an oil-in-water emulsion, a water-in-oil emulsion, a solid anhydrous gel, a soft anhydrous gel, a loose powder, a compacted powder, or a two-phase form. The compositions described herein may be in the form of a composition with an oily and, in one embodiment, anhydrous continuous phase; in this embodiment, it may comprise an aqueous phase in an amount of less than 5%.

The composition described herein may be in the form of a colored skincare composition, uncolored skincare composition, antisun composition, makeup-removing composition, or hygiene composition. In embodiments wherein the composition comprises cosmetic active agents, the composition may be used as a non-therapeutic care or treatment base for the skin such as for the hands, face, lips (e.g., lip balms, for protecting the lips against cold, sunlight, and/or the wind), or may be a product for artificially tanning the skin.

The composition described herein may also be in the form of a colored skin makeup product, such as for the face, for example a blusher, makeup rouge, eyeshadow, body makeup product (for example, a semi-permanent tattoo product), lip makeup product (for example, a lipstick or a lip gloss, possibly having non-therapeutic care or treatment properties), makeup product for the integuments (for example, nail polish, mascara, eyeliner, hair coloring products, and haircare products).

In one embodiment, the composition according to the present disclosure is in the form of a lipstick or a lip gloss.

Needless to say, the composition of the present disclosure should be physiologically acceptable (such as cosmetically acceptable), i.e. it should be non-toxic and able to be applied to human skin, integuments, and lips.

The term “cosmetically acceptable,” as used herein, is understood to mean having a pleasant taste, feel, appearance and/or odor, which may be applied for several days over several months.

The composition according to the disclosure may be manufactured via the known processes generally used in cosmetics.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

The examples that follow are intended to illustrate, in a non-limiting manner, the subject of the present invention. Amounts below are given as mass percentages.

### EXAMPLES

### Example 1

### Stick of Lipstick

| | Amount in |
|---|---|
| Chemical name | grams |
| Ethers of dodecanediol and of polyethylene glycol | 10.3 |
| Octyldodecyl PPG-3 myristyl ether dimer dilinoleate | 22.15 |
| (Liquiwax PolyEFA) | |
| Isostearyl neopentanoate | 30.3 |
| Isocetyl stearoyl stearate | 20.6 |
| Polyvinyl laurate | 16.5 |
| Di-tert-butyl 4-hydroxytoluene | 0.15 |
| Vinyl acetate/allyl stearate copolymer | 7.5 |
| Hydrogenated polyisobutene | 10 |
| Polybutene/polyisobutene copolymer | 10 |
| Polyethylene wax | 3.4 |
| Microcrystalline wax | 2.55 |
| Octacosanyl stearate | 4.25 |
| Esters of linear acids and alcohols | 3.4 |
| Alumina/silica/trimethylolpropane-treated rutile titanium oxide | 0.27 |
| Aluminium lake of the disodium salt of phloxin B on alumina, | 0.66 |
| aluminium benzoate | |
| Calcium salt of lithol B red | 0.16 |
| Brown, yellow iron oxides | 0.64 |
| Black iron oxide | 0.66 |
| Polydimethylsiloxane-coated porous silica microspheres | 5 |
| Fragrance | 0.4 |

The mean gloss values for this lipstick T0h and T5h measured at 60°, according to the method described above, were found to be equal to 79 out of 100.

### Example 2

### Stick of Lipstick

| CHEMICAL NAME | (weight %) |
|---|---|
| Octyldodecanol | 14.7 |
| Phenyl trimethicone (DC 556 from Dow Corning, 20 cSt) | 4.21 |
| Diisostearyl malate | 12.88 |
| Pentaerythrityl tetraisostearate | 10.31 |
| Hydrogenated castor oil isostearate | 9.49 |
| Bis-diglyceryl polyacyladipate-2 | 9.49 |
| Stearyl/PPG-3 myristyl ether dimer dilinoleate (Liquiwax | 9.49 |
| PolyIPL) | |
| C18-36 acid triglyceride | 0.71 |
| Preserving agent | 0.06 |
| Microcrystalline wax (Microwax HW from Paramelt) | 3.75 |
| Hydrogenated coco-glycerides | 6.25 |
| Polyethylene wax (Performalene 500 polyethylene from | 10 |
| NPT) | |
| Pigments | 8.66 |

The mean gloss values T0h and T5h of the composition measured at 60° according to the method described above were found to be equal to 72 out of 100.

What is claimed is:

1. A cosmetic composition comprising:
at least one alkoxylated ester derived from at least one alkoxylated alcohol
and at least one carboxylic acid; and
at least one apolar oil,
wherein when the composition forms a deposit, the deposit has a mean gloss, measured at 60°, of greater than or equal to 45 out of 100.

2. A cosmetic composition comprising:
at least one alkoxylated ester derived from at least one alkoxylated alcohol
and at least one carboxylic acid; and
at least one apolar oil,
wherein the at least one alkoxylated ester is chosen from polyesters in which only one ester functional group is obtained by reacting an acid functional group of a polycarboxylic acid with an alkoxylated alcohol.

3. The cosmetic composition according to claim 1, wherein the at least one alkoxylated ester is obtained by reacting a monocarboxylic acid with a polyalkoxylated alcohol.

4. The cosmetic composition according to claim 3, wherein the at least one alkoxylated ester is chosen from the compounds of Formula (II): 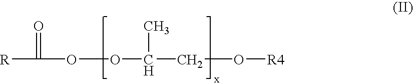
wherein:
x is an integer ranging from 2 to 40;
R₄ is chosen from saturated and unsaturated, substituted and unsubstituted, aliphatic hydrocarbon-based groups comprising from 3 to 36 carbon atoms, and
RCOO is chosen from:
monocarboxylic acid residues; and
aromatic acid residues comprising a benzenyl ring optionally substituted with a group chosen from OH, NH₂, methyl and ethyl groups.

5. The cosmetic composition according to claim 4, wherein x is an integer ranging from 3 to 30.

6. The cosmetic composition according to claim 5, wherein x is an integer ranging from 3 to 10.

7. The cosmetic composition according to claim 4, wherein R₄ is chosen from saturated and unsaturated, substituted and unsubstituted, aliphatic hydrocarbon-based groups comprising from 4 to 24 carbon atoms.

8. The cosmetic composition according to claim 7, wherein R₄ is chosen from saturated and unsaturated, substituted and unsubstituted, aliphatic hydrocarbon-based units comprising from 6 to 18 carbon atoms.

9. The cosmetic composition according to claim 4, wherein RCOO is a monocarboxylic acid residue of formula (R₂R₃R₄C)COO wherein R₂, R₃ and R₄ are chosen, independently from each other, from methyl, ethyl, propyl and isopropyl groups.

10. The cosmetic composition according to claim 4, wherein RCOO is a monocarboxylic acid chosen from 2-ethylhexanoic acid, caproic acid, neopentanoic acid, isostearic acid, neoheptanoic acid, and oleic acid.

11. The cosmetic composition according to claim 3, wherein the at least one ester obtained by reacting a monocarboxylic acid with a polyalkoxylated alcohol is chosen from PPG-3 myristyl ether neoheptanoate and PPG-4 butyloctyl ether ethylhexanoate.

12. The cosmetic composition according to claim 1, wherein the at least one alkoxylated ester is chosen from the polyesters obtained by reacting at least one polycarboxylic acid with a stoichiometric excess of at least one alkoxylated alcohol relative to the number of acid functional groups in the acid.

13. The cosmetic composition according to claim 12, wherein the at least one alkoxylated alcohol comprises an alkyl chain comprising from 8 to 36 carbon atoms.

14. The cosmetic composition according to claim 13, wherein the at least one alkoxylated alcohol comprises an alkyl chain comprising from 10 to 22 carbon atoms.

15. The cosmetic composition according to claim 12, wherein the at least one alkoxylated alcohol comprises a myristyl chain.

16. The cosmetic composition according to claim 12, wherein the at least one polycarboxylic acid is a dicarboxylic acid chosen from aliphatic dicarboxylic acids comprising from 2 to 36 carbon atoms.

17. The cosmetic composition according to claim 16, wherein the at least one polycarboxylic acid is a dicarboxylic acid chosen from adipic acid, malonic acid succinic acid, phthalic acid, and maleic acid.

18. The cosmetic composition according to claim 1, wherein the at least one alkoxylated ester is a polyester wherein only one ester functional group is obtained by reacting an acid functional group of a polycarboxylic acid with an alkoxylated alcohol.

19. The cosmetic composition according to claim 1, wherein the at least one alkoxylated ester is chosen from the compounds of Formula (III): 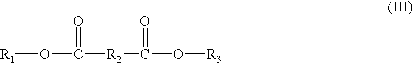
wherein R₁ is chosen from compounds of Formula (IV): 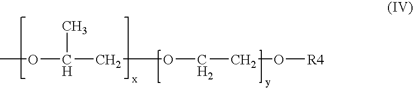
wherein:
R₄ is chosen from saturated and unsaturated, substituted and unsubstituted, aliphatic groups comprising from 4 to 24 carbon atoms;
x is an integer ranging from 3 to 30;
y is an integer ranging from 3 to 30;
R₂ is chosen from saturated and unsaturated, substituted and unsubstituted, aliphatic units comprising from 4 to 40 carbon atoms; and
R₃ is chosen from saturated and unsaturated, straight-chain and branched-chain, aliphatic units comprising from 4 to 32 carbon atoms.

20. The cosmetic composition according to claim 19, wherein R₃ is chosen from saturated and unsaturated, straight-chain and branched-chain, aliphatic groups comprising from 12 to 24 carbon atoms.

21. The cosmetic composition according to claim 19, wherein R₄ is chosen from saturated aliphatic groups comprising from 12 to 20 carbon atoms.

22. The cosmetic composition according to claim 19, wherein R₂ is chosen from saturated aliphatic groups comprising from 4 to 40 carbon atoms.

23. The cosmetic composition according to claim 19, wherein x and y are independently of each other, chosen from 3 and 4.

24. The cosmetic composition according to claim 19, wherein R₃ is chosen from saturated branched-chain aliphatic groups comprising from 12 to 20 carbon atoms.

25. The cosmetic composition according to claim 24, wherein R₃ is chosen from octyldodecyl, isostearyl and stearyl.

26. The cosmetic composition according to claim 19, wherein the at least one alkoxylated ester is octyldodecyl PPG-3 myristyl ether dimer dilinoleate.

27. The cosmetic composition according to claim 19, wherein the at least one alkoxylated ester is isostearyl PPG-4 butyloctyl ether dimer dilinoleate.

28. The cosmetic composition according to claim 1, wherein the at least one apolar oil is such that, when it is present in a sufficient amount in the composition, and when the composition forms a deposit, the deposit has a mean gloss greater than or equal to 45 out of 100.

29. The cosmetic composition according to claim 1, wherein the at least one apolar oil is non-volatile.

30. The cosmetic composition according to claim 1, wherein the at least one apolar oil is chosen from hydrocarbon-based oils.

31. The cosmetic composition according to claim 1, wherein the at least one apolar oil is free of heteroatoms.

32. The cosmetic composition according to claim 31, wherein the at least one apolar oil is chosen from hydrocarbon-based oils.

33. The cosmetic composition according to claim 32, wherein the at least one apolar hydrocarbon-based oil is chosen from linear and branched saturated alkanes.

34. The cosmetic composition according to claim 1, wherein the at least one apolar oil is chosen from apolar hydrocarbon-based oils with a molecular mass ranging from 300 g/mol to 900 g/mol.

35. The cosmetic composition according to claim 34, wherein the at least one apolar oil is chosen from apolar hydrocarbon-based oils with a molecular mass ranging from 350 g/mol to 800 g/mol.

36. The cosmetic composition according to claim 29, wherein the at least one non-volatile hydrocarbon-based apolar oil is chosen from linear and branched hydrocarbons.

37. The cosmetic composition according to claim 36, wherein the at least one non-volatile hydrocarbon-based apolar oil is chosen from liquid paraffin, liquid petroleum jelly, naphthalene oil, hydrogenated polyisobutene, polybutene/polyisobutene copolymers, isoeicosane, squalane, polydecenes, and decene/butene copolymers.

38. The cosmetic composition according to claim 1, wherein the mean gloss of the composition T0h, once spread onto a support, measured at 60°, is greater than or equal to 45 out of 100.

39. The cosmetic composition according to claim 38, wherein the mean gloss of the composition T0h, once spread onto a support, measured at 60°, is greater than or equal to 75 out of 100.

40. The cosmetic composition according to claim 38, wherein the mean gloss of the composition T5h, once spread onto a support, measured at 600, is greater than or equal to 35 out of 100.

41. The cosmetic composition according to claim 40, wherein the mean gloss of the composition T5h, once spread onto a support, measured at 600, is greater than or equal to 75 out of 100.

42. The cosmetic composition according to claim 40, wherein the mean loss of gloss over time, expressed as the ratio (T0h−T5h)/T0h, is less than or equal to 25%.

43. The cosmetic composition according to claim 42, wherein the mean loss of gloss over time, expressed as the ratio (T0h−T5h)/T0h, is less than or equal to less than or equal to 5%.

44. The cosmetic composition according to claim 1, further comprising at least one dyestuff chosen from water-soluble dyes, liposoluble dyes, and pulverulent dyestuffs.

45. The cosmetic composition according to claim 44, wherein the at least one pulverulent dyestuff is chosen from pigments, nacres, and flakes

46. The cosmetic composition according to claim 1, further comprising at least one fatty substance chosen from waxes and pasty fatty substances.

47. The cosmetic composition according to claim 1, further comprising at least one cosmetic adjuvant chosen from vitamins, thickeners, film-forming agents, gelling agents, trace elements, softeners, sequestering agents, fragrances, acidifying agents, basifying agents, preserving agents, sunscreens, surfactants, antioxidants, fibers, agents for preventing hair loss, eyelash care agents, antidandruff agents, and propellants.

48. The cosmetic composition according to claim 1, wherein the composition is in the form of a suspension, dispersion, solution, gel, emulsion, cream, paste, mousse, dispersion of vesicles, two-phase lotion, multiphase lotion, spray, powder, stick, or cast solid.

49. The cosmetic composition according to claim 1, wherein the composition is in anhydrous form.

50. A cosmetic composition comprising at least one ester derived from at least one alkoxylated alcohol and at least one carboxylic acid;
and at least one apolar oil,
wherein when the composition forms a deposit, the deposit has a mean gloss, measured at 60°, of greater than or equal to 30 out of 100, and wherein the composition is free of lanolin and derivatives thereof.

51. A cosmetic composition according to claim 1, wherein the composition is a makeup or care composition for keratin materials.

52. The cosmetic composition according to claim 1, wherein it is in the form of a lip makeup product.

53. A process for making up and/or caring for the skin, lips, and/or integuments, comprising applying to the skin, lips and/or integuments at least one composition comprising:
at least one alkoxylated ester derived from at least one alkoxylated alcohol and at least one carboxylic acid; and
at least one apolar oil,
wherein when the composition forms a deposit, the deposit has a mean gloss, measured at 60°, of greater than or equal to 45 out of 100.
